(19) United States
(12) Patent Application Publication
(10) Pub. No.: US 2020/0375530 A1
(43) Pub. Date: Dec. 3, 2020

(54) SYSTEMS AND METHODS FOR MONITORING BRAIN HEALTH

(71) Applicant: EpilepsyCo Inc., Guilford, CT (US)

(72) Inventors: Eric Kabrams, Redwood City, CA (US); Kamyar Firouzi, San Jose, CA (US)

(73) Assignee: EpilepsyCo Inc., Guilford, CT (US)

(21) Appl. No.: 16/886,408

(22) Filed: May 28, 2020

Related U.S. Application Data
(60) Provisional application No. 62/854,107, filed on May 29, 2019.
(60) Provisional application No. 62/854,113, filed on May 29, 2019.

(51) Int. Cl. A61B 5/00

(52) U.S. Cl. CPC A61B 5/4836; A61B 5/4094; A61B 5/0017; A61B 5/4088; A61B 5/7275; A61B 5/4064

(57) ABSTRACT

In some aspects, a closed-loop system for monitoring and/or treating brain function of a person includes a CMUT device that transmits ultrasound radiation to the brain of the person and receives and detects an ultrasound signal generated in the brain, and a processor that assesses the received ultrasound signal to determine brain function and controls further transmission of ultrasound radiation from the CMUT device based on the determined brain function.

### CROSS REFERENCE TO RELATED APPLICATIONS

This application claims priority under 35 U.S.C. § 119(e) to U.S. Provisional Application Ser. No. 62/854,107, titled “SENSORS FOR MONITORING BRAIN HEALTH,” filed May 29, 2019 and U.S. Provisional Application Ser. No. 62/854,113, titled “SYSTEM FOR MONITORING BRAIN HEALTH,” filed May 29, 2019, both of which are hereby incorporated herein by reference in their entireties.

### BACKGROUND

Neurological disorders affecting brain health constitute a significant portion of the global burden of disease. Such disorders can include epilepsy, Alzheimer's disease, and Parkinson's disease. For example, about 65 million people worldwide suffer from epilepsy. In the developing world, onset is more common in older children and young adults, due to differences in the frequency of the underlying causes. Nearly 80% of cases occur in the developing world. In the developed world, onset of new cases occurs most frequently in babies and the elderly. The United States itself has about 3.4 million people suffering from epilepsy with an estimated $15 billion economic impact. These patients suffer from symptoms such as recurrent seizures, which are episodes of excessive and synchronized neural activity in the brain. In many areas of the world, those with epilepsy either have restrictions placed on their ability to drive or are not permitted to drive until they are free of seizures for a specific length of time.

### SUMMARY

The methods/devices described herein provide for monitoring brain conditions as well as functions using opto-acoustic phenomena in a manner that is noninvasive (or minimally invasive), and, in some cases, wireless, and continuous as well. Brain functions, to be diagnosed and monitored, include but are not limited to detection of epileptic seizure and neuroimaging. Brain conditions, to be diagnosed and monitored, include but are not limited to brain tumors, stroke, traumatic brain injury, vasospasm, change in intracranial pressure, hydrocephalus, and surgical cavity. In some embodiments, an ultrasonic transducer in the receive mode is utilized in various form factors including wearable as well as implantable forms to receive the acoustic waves that are generated by electromagnetic energy such as an optical source or antenna. Depending on the number of opto-acoustic devices, in some embodiments, the systems and methods described herein may be used for detection as well as imaging. Additionally or alternatively, the systems and methods described herein may be used for diagnostic and/or monitoring purposes.

In some aspects, the described systems and methods provide for a sensor comprising an optical transmitter that transmits to a brain of a person infrared radiation and an acoustic receiver that detects an ultrasound signal generated in the brain in response to the infrared radiation.

In some embodiments, the sensor further includes a processor that determines oxygen saturation of the brain based on the detected ultrasound signal.

In some embodiments, the oxygen saturation of the brain is indicative of neural activity in the brain.

In some embodiments, the processor implements a statistical model to predict, based on the oxygen saturation, whether a seizure will occur in the brain.

In some embodiments, the optical transmitter includes an array of at least one of light emitting diodes (LEDs), lasers, and laser diodes.

In some embodiments, the acoustic receiver includes at least one capacitive micromachined ultrasound transducer (CMUT).

In some embodiments, the acoustic receiver includes an array of CMUTs.

In some embodiments, the array of at least one of LEDs, lasers and laser diodes is intermixed with the array of CMUTs.

In some aspects, the described systems and methods provide for a method comprising transmitting to the brain of a person infrared radiation and detecting an ultrasound signal generated in the brain in response to the infrared radiation.

In some embodiments, the method further includes determining oxygen saturation of the brain based on the detected ultrasound signal.

In some embodiments, the oxygen saturation of the brain is indicative of neural activity in the brain.

In some embodiments, the method further includes using a statistical model to predict, based on the oxygen saturation, whether a seizure will occur in the brain.

In some embodiments, the transmitting is performed by an array of at least one of LEDs, lasers, and laser diodes.

In some embodiments, the detecting is performed by at least one CMUT.

In some aspects, the described systems and methods provide for a system for monitoring and/or treating brain function of a person comprising a sensor, including an optical transmitter that transmits to the brain of the person infrared radiation and an acoustic receiver that detects an ultrasound signal generated in the brain in response to the infrared radiation, a cap to be worn by the person and/or patches to be placed on the person, the sensor being located within the cap and/or patches.

In some aspects, the described systems and methods provide for a closed-loop system for monitoring and/or treating brain function of a person comprising a capacitive micromachined ultrasound transducer (CMUT) device that transmits ultrasound radiation to the brain of the person and receives and detects an ultrasound signal generated in the brain of the person and a processor that assesses the received ultrasound signal to determine brain function and controls further transmission of ultrasound radiation from the CMUT based on the determined brain function.

In some embodiments, assessing the received ultrasound signal to determine brain function comprises assessing a likelihood of a seizure of the person, and controlling further transmission of ultrasound radiation from the CMUT device comprises controlling further transmission of ultrasound radiation from the CMUT device when the likelihood is greater than a certain threshold.

In some embodiments, the system includes an array of CMUT devices.

In some embodiments, the CMUT device transmits at, near or below 1 MHz.

In some embodiments, the processor implements a statistical model to predict, based on the received ultrasound signal, whether a seizure will occur in the brain.

In some embodiments, the system includes an optical transmitter that transmits to the brain infrared radiation.

In some embodiments, the CMUT device receives and detects the ultrasound signal generated in the brain in response to the infrared radiation.

In some embodiments, determining brain function comprises determining oxygen saturation of the brain based on the received ultrasound signal, wherein the oxygen saturation of the brain is indicative of neural activity in the brain.

In some aspects, the described systems and methods provide for a method of monitoring and/or treating brain function of a person comprising transmitting with a CMUT device ultrasound radiation to the brain of the person, receiving an ultrasound signal generated in the brain of the person, determining brain function based on the received ultrasound signal, and controlling further transmission of ultrasound radiation from the CMUT device based on the determined brain function.

In some embodiments, assessing the received ultrasound signal to determine brain function comprises assessing a likelihood of a seizure of the person, and controlling further transmission of ultrasound radiation from the CMUT device comprises controlling further transmission of ultrasound radiation from the CMUT device when the likelihood is greater than a certain threshold.

In some embodiments, determining is performed by a processor implementing a statistical model to predict, based on the received ultrasound signal, whether a seizure will occur in the brain.

In some embodiments, the method further includes transmitting to the brain infrared radiation.

In some embodiments, the received ultrasound signal is generated in the brain of the person in response to the infrared radiation.

In some embodiments, determining brain function comprises determining oxygen saturation of the brain based on the received ultrasound signal.

In some embodiments, the oxygen saturation of the brain is indicative of neural activity in the brain.

While the sensors, systems, and methods described herein can be used to monitor and/or treat epilepsy as described, the sensors, systems, and methods are not so limited. For example, the sensors, systems and methods can be used to monitor and/or treat general brain function and/or other brain conditions.

### BRIEF DESCRIPTION OF THE DRAWINGS

Various aspects and embodiments will be described with reference to the following figures. The figures are not necessarily drawn to scale.

FIG. 1 shows an illustrative example of an opto-acoustic sensor, in accordance with some embodiments of the technology described herein.

FIG. 2 shows another illustrative example of an opto-acoustic sensor, in accordance with some embodiments of the technology described herein.

FIG. 3 shows yet another illustrative example of an opto-acoustic sensor, in accordance with some embodiments of the technology described herein.

FIG. 4 shows yet another illustrative example of an opto-acoustic sensor, in accordance with some embodiments of the technology described herein.

FIG. 5 shows yet another illustrative example of an opto-acoustic sensor, in accordance with some embodiments of the technology described herein.

FIG. 6 shows yet another illustrative example of an opto-acoustic sensor, in accordance with some embodiments of the technology described herein.

FIG. 7 shows yet another illustrative example of an opto-acoustic sensor, in accordance with some embodiments of the technology described herein.

FIG. 8 shows an illustrative example of sensor placement, in accordance with some embodiments of the technology described herein.

FIG. 9 shows a convolutional neural network that may be used to detect and/or predict one or more symptoms of a neurological disorder, in accordance with some embodiments of the technology described herein.

FIG. 10 shows an illustrative flow diagram for a process for brain monitoring and treatment, in accordance with some embodiments of the technology described herein.

FIG. 11 shows a block diagram of an illustrative computer system that may be used in implementing some embodiments of the technology described herein.

### DETAILED DESCRIPTION

The inventors have appreciated that conventional approaches toward monitoring and/or treating brain conditions suffer from significant limitations, including cost, invasiveness, and lack of effectiveness. In some aspects, devices, methods, and systems are disclosed herein to monitor brain conditions and function non-invasively or minimally invasively. Such devices, methods, and systems in some embodiments also diagnose and/or treat brain conditions.

For example, the described systems and methods may be used to treat epilepsy, which is a group of neurological disorders characterized by epileptic seizures. Epileptic seizures are episodes that can vary from brief and nearly undetectable periods to long periods of vigorous shaking. These episodes can result in physical injuries, including occasionally broken bones. In epilepsy, seizures tend to recur and have no immediate underlying cause. Some cases occur as the result of brain injury, stroke, brain tumors, infections of the brain, and birth defects through a process known as epileptogenesis. In such cases, epileptic seizures are the result of excessive and abnormal brain function, including abnormal neuronal activity in the cortex of the brain. The diagnosis involves ruling out other conditions that might cause similar symptoms, such as fainting, and determining if another cause of seizures is present, such as alcohol withdrawal or electrolyte problems. This may be partly done by imaging the brain and performing blood tests. The diagnosis of epilepsy is typically made based on observation of the seizure onset and the underlying cause. A functional neuroimaging method, such as Electroencephalography (EEG), to look for abnormal patterns of brain function, including brain waves, and a structural neuroimaging method, such as Computed Tomography (CT) or Magnetic Resonance Imaging (MRI), to look at the structure of the brain are also usually part of the diagnosis. Epilepsy usually cannot be cured, unless surgery is performed. However, the outcome of surgery can lead to unexpected harsh outcomes such as loss of functionality of certain abilities such as speech, control over movements, etc.

The inventors herein have recognized limitations with existing methods, systems, and devices for monitoring and treating brain function. Conventional non-invasive technology includes EEG and MRI/CT scans. EEG is an electrophysiological monitoring method to record electrical activity of the brain. EEG is typically noninvasive, with the electrodes placed along the scalp, although invasive electrodes are sometimes used such as in electrocorticography. EEG measures voltage fluctuations resulting from ionic current within the neurons of the brain. EEG is most often used to diagnose epilepsy, which causes abnormalities in EEG readings. EEG has poor spatial resolution for diagnosis. Often for proper diagnosis or detection of epilepsy both high temporal resolution and spatial resolution is required. To capture the structure of the nervous system and the diagnosis of large scale intracranial disease (such as tumor) or injury, and for detection of epileptic events, MRI and CT can be used. They provide good spatial resolution for diagnosis. However, they have poor temporal resolution. Moreover, they are very expensive and not portable. Despite limited spatial resolution, EEG is one of the few portable techniques available and offers millisecond-range temporal resolution which is not possible with CT or MRI. Moreover, early detection of disease recurrence and infra clinic brain changes requires a continuous imaging and/or monitoring system, which is especially important for early recurrence detection (e.g., for brain tumors).

Using Opto-Acoustic Sensors to Predict Seizures and/or Functional Imaging of the Brain

The inventors have discovered that application of ultrasound and/or optical imaging in conventional systems has been limited because of the presence of the skull, which is attenuating to both ultrasound as well as optical energy, primarily due to a highly heterogeneous structure that can scatter both optical and sound waves, sometimes dramatically. The inventors have also discovered that preceding a seizure there may be substantial changes away from the baseline value of the oxygen saturation of cerebral tissue. Such changes can be measured near the focus of the seizure and/or in arteries at a distance from the seizure focus, e.g., several centimeters or another suitable distance. For example, cerebral oxygen saturation may be measured using Near Infrared Spectroscopy (NIRS). An infrared transmitter sends infrared radiation into a target body of interest and a receiver present at a second location measures the infrared signal, where the receiver is placed at a suitably selected distance from the transmitter. Of the radiation that is emitted by the transmitter, some is absorbed by the tissue and some is not absorbed by the tissue. Of the radiation that is not absorbed by the tissue, some takes a path which terminates at the location of the optical receiver (and is therefore recorded by the optical receiver), and some is scattered to other locations. The signal recorded by the optical receiver (e.g., the NIRS signal) is therefore related to the amount of radiation that is absorbed by the tissue: the more radiation that is absorbed by the tissue, the lower the magnitude of the NIRS signal. This may lead to a contrast in blood oxygenation and possibly other markers that can act as a viable photoacoustic contrast mechanism.

In some aspects, the inventors have designed an opto-acoustic sensor that uses photoacoustic spectroscopy to measure oxygen saturation. In photoacoustic spectroscopy, an optical transmitter (e.g., for visible, infrared, or ultraviolet wavelengths) sends radiation of a known wavelength into a sample, and an acoustic receiver measures the resulting acoustic response. The magnitude of the acoustic response is used to deduce the concentration of the absorbing compound in the sample. Opto-acoustics includes physical processes in which the absorption of the electromagnetic energy at various wavelengths acts as a source of sound via rapid thermal expansion (and/or contraction) of the illuminated tissue. For example, the atoms in the receiving material may absorb the electromagnetic radiation and get excited to higher energy levels. Over short time scales (e.g., nanoseconds), the atoms may radiate this extra energy away as heat. The ensuing local changes in temperature may cause the local volume of the material to expand and contract, causing a transient increase and decrease in pressure. This transient pressure-change initiates a traveling pressure-wave that propagates through the surrounding medium (typically in a manner that is dependent on the mechanical properties of the medium).

Often, when visible light or near infrared (NIR) wavelengths are applied, it is referred to as the photoacoustic (PA)/opto-acoustic (OA) effect, and when radio-frequency waves are used, it is referred to as the thermoacoustic (TA) effect. Herein, PA or OA are interchangeably used to mean the same effect. PA can overcome the disadvantage of optical imaging in spatial resolution and the disadvantage of ultrasound imaging in contrast and speckle artifact. The PA effect is the generation of acoustic waves by the absorption of electromagnetic energy such as optical or radiofrequency waves. Thus, for PA images optical absorption rate is the main contrast mechanism. The main contrast mechanism between blood vessels and the surrounding tissue in photoacoustic/opto-acoustic imaging is optical energy absorption by hemoglobin in the blood, which is directly proportional to the oxygen concentration in the blood. The main contrast mechanism in thermoacoustic imaging (by utilizing the radiofrequency waves) is water content. The absorption rate is a function of the optical wavelength (or frequency) and thus a multi-color (multi-wavelength) source can be used to achieve contrast between various tissue structures.

In some aspects, the inventors have designed an opto-acoustic sensor that uses photoacoustic spectroscopy to measure oxygen saturation with a higher signal-to-noise ratio than what can be achieved with NIRS alone. There are several factors that lead to the improved performance. The inventors have recognized that photoacoustic spectroscopy records a signal that is more directly related to oxygen saturation than that recorded by NIRS. In some embodiments, NIRS and photoacoustic spectroscopy are complementary, where the NIRS signal is due to the transmitted IR radiation that is not absorbed by the tissue and is not scattered away from the optical detector. The NIRS signal therefore depends on both the absorption of the radiation by the molecules of interest, and the scattering of the radiation by the entire tissue volume, which can be quite complex and convoluted. In contrast, the photoacoustic signal is due only to the transmitted IR radiation that is absorbed by the tissue. Therefore, the photoacoustic signal is related solely to the concentration of the absorbing molecules of interest. The photoacoustic measurement is therefore a more direct measurement of the quantity of interest.

In some embodiments, pressure generated in photoacoustic excitation is directly proportional to optical fluence, which is a measure of input energy, and absorption rate through the following equation,

Pressure=C*mu_a*phi, where:
phi represents optical fluence,
mu_a represents an optical absorption coefficient, and
C represents a constant representing efficiency of converting heat energy (as a result of optical absorption) into acoustic pressure.

The optical fluence may be a variable controlled by the optical transmitter. The optical absorption coefficient may be determined by measuring the acoustic pressure on the ultrasound receiver. If more than one wavelength is chosen, the absorption spectrum (i.e., absorption as a function of optical wavelength) may also be calculated. Some tissue types such as blood with a specific chemical composition may have known absorption spectra. Blood may have the most significant photoacoustic contrast among different tissue types. This contrast may be directly correlated to the amount of its oxygen content, where higher concentration may indicate stronger photoacoustic pressure. As such, by measuring and monitoring the changes in the absorption spectrum, one can estimate the changes in the oxygen content of blood and thereby estimate the flow of the oxygenated blood, which can be an indicator of strong neural activity such as seizure.

The inventors have further recognized that photoacoustic spectroscopy can achieve significantly better spatial resolution at locations significantly deeper in the brain (e.g., 5-10 cm or another suitable distance) than that obtained by NIRS at comparable depths. The spatial resolution of NIRS is solely dependent on the spatial resolution of the irradiating light. When transmitting through the skull, significant scattering occurs, making it extremely difficult to focus light beyond a few millimeters deep into the brain tissue. In contrast, in photoacoustic sensing, the spatial resolution can come from either the spatial resolution of the irradiating light, or from operating the acoustic receiver as a phased-array, a technique commonly referred to as beamforming. The latter is done by having an array of acoustic receivers, each of which can be read-out independently. To record only the sound coming from a particular location in space, the signals of the individual acoustic receivers are each delayed by a particular amount and then linearly combined with particular weights. The weight and time-delay applied to the signal at each acoustic sensor is dependent on the geometry of the receiver array and the location of the intended focal-point. By using the acoustic receiver, rather than the optical transmitter, to determine the spatial resolution of the system, signals can be read up to about 5-10 centimeters deep from the surface of the brain. Thus, photoacoustic spectroscopy can be used to record with high spatial-resolution from locations far deeper in the brain. This may enable measuring oxygen saturation in major cerebral arteries that are too deep for NIRS to measure from.

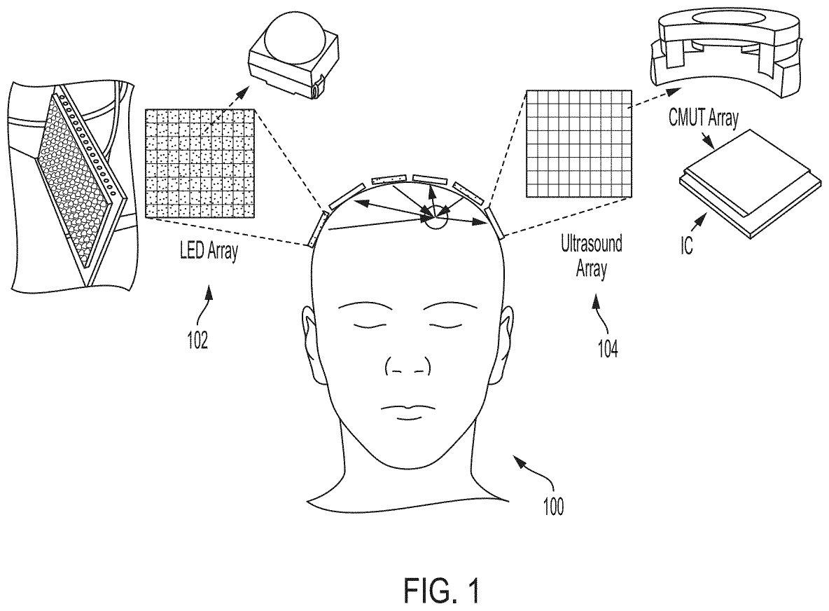

FIG. 1 shows an embodiment of an opto-acoustic sensor described herein. The opto-acoustic sensor 100 features an optical transmitter 102 (e.g., operating in the infrared or visible range), such as an LED array, a laser, or a laser diode, or other suitable devices, and an acoustic receiver 104, such as a capacitive micromachined ultrasound transducer (CMUT) or a piezoelectric crystal or other suitable devices. The acoustic receiver may be a single-element receiver, or may be a 1D or 2D array, placed out in a geometry such as a grid, a ring, a curved surface, or another suitable shape. The optical transmitter transmits infrared radiation at one, two, or several different wavelengths, each modulated at a frequency at or around 500 kHz, which causes the blood in the brain tissue to generate ultrasound waves at 500 kHz, which can pass through the skull with significantly less attenuation than what would occur at higher carrier frequencies. The magnitude of the received ultrasound signals may indicate the oxygen saturation of the tissue of interest. As discussed above, this is because optical energy absorption by hemoglobin in the blood may be directly proportional to the oxygen concentration in the blood. The sensor can focus on particular locations within the brain, with a resolution of, e.g., 10s of cubic millimeters, by using beam forming algorithms on the acoustic array. For target locations near the surface of the brain, the focusing may be achieved using a combination of focusing the light and beam-forming algorithms on the acoustic array.

In some embodiments, the acoustic receiver includes a CMUT with a flexible top plate suspended over a gap, forming a variable capacitor. The displacement of the top plate may create an acoustic pressure in the medium (or vice versa), where the acoustic pressure in the medium displaces the flexible plate. Transduction may be achieved electrostatically, by converting the displacement of the flexible plate to an electric current through modulating the electric field in the gap, in contrast with piezoelectric transducers. The merit of the CMUT derives from having a very large electric field in the cavity of the capacitor, a field of the order of 10\hat{ }8 V/m or higher, resulting in an electro-mechanical coupling coefficient that competes with the best piezoelectric materials. The availability of micro-electro-mechanical-systems (MEMS) technologies makes it possible to realize thin vacuum gaps where such high electric fields can be established with relatively low voltages. Thus, viable CMUT-based devices can be realized and even integrated directly on electronic circuits such as complimentary metal-oxide-semiconductor (CMOS).

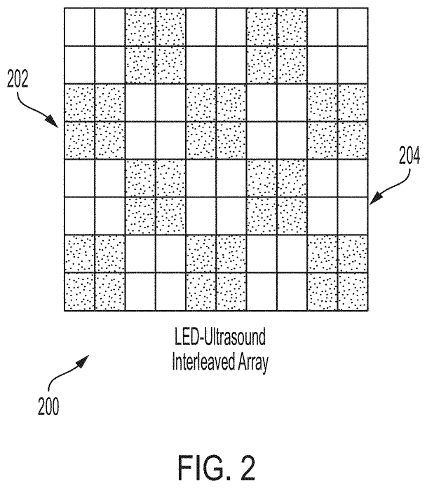

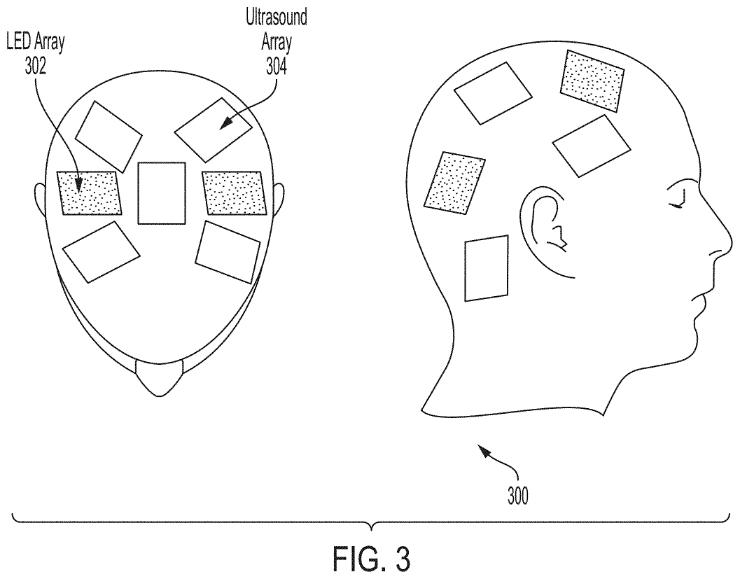

The inventors have developed several embodiments, including the sensors, for monitoring and/or treating brain function. For example, some embodiments are non-invasive, while others are minimally invasive. Some embodiments of the sensors, including placement on the scalp, are shown in FIGS. 2 and 3. In some embodiments, an array of optical transmitter elements (e.g., light emitting diodes (LEDs)) is intermixed with an array of acoustic receiver elements (e.g., CMUTs). FIG. 2 shows an example 200 where an array of optical transmitter elements 202 (e.g., LEDs) is intermixed with an array of acoustic receiver elements 204 (e.g., CMUTs). In some embodiments, the array of optical transmitter elements is separate from the array of acoustic receiver elements. FIG. 3 shows an example 300 where an array of optical transmitter elements 302 is separate from an array of acoustic receiver elements 304.

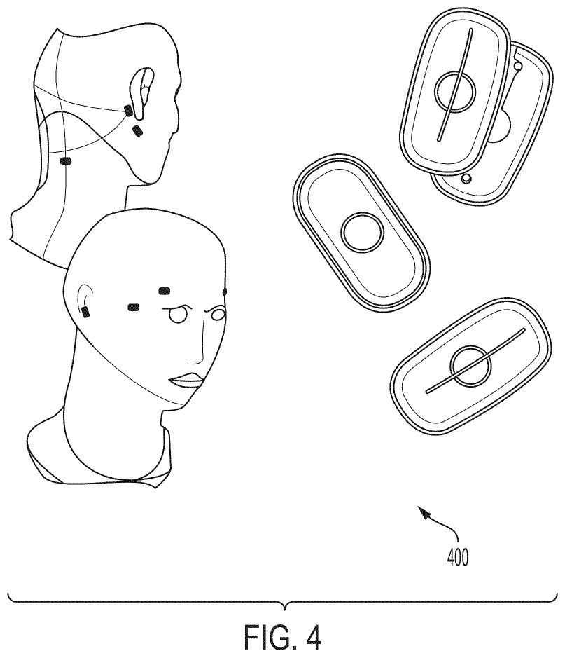

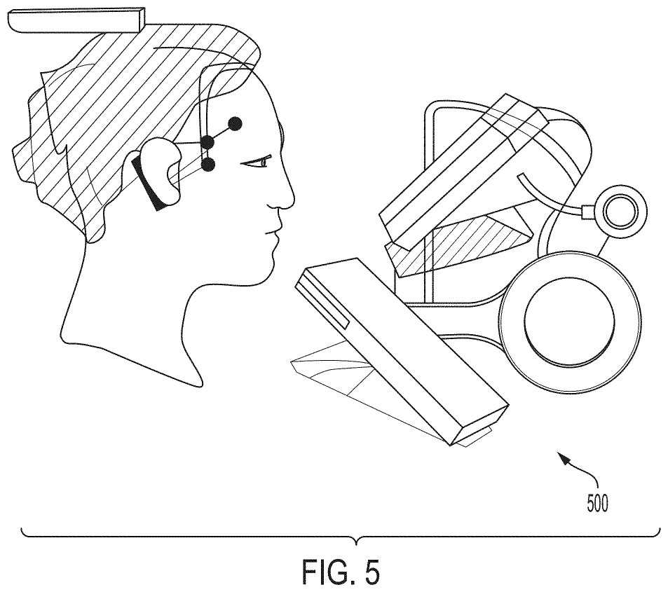

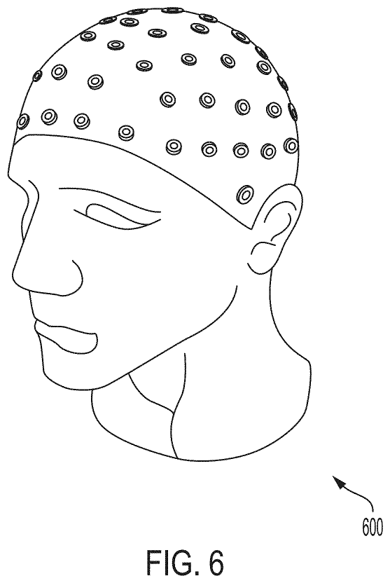

In some embodiments, a non-invasive configuration includes an integrated opto-acoustic device that illuminates the brain and receives the ultrasound waves noninvasively and trans-cranially. In the configuration that includes few (between one and several) opto-acoustic sensors, the form factor for the device may be one or several small adhesive patches. FIGS. 4 and 5 show examples 400 and 500 of such configurations. In the configuration that includes many opto-acoustic sensors, the form-factor may be similar to a helmet, a hat, or another suitable form. The device may include many optical sources (such as LEDs) and acoustic/ultrasound sensors that are interleaved together and integrated with the application-specific integrated circuit (ASIC) inside the helmet. The opto-acoustic elements can be wirelessly charged and transfer data to a hub that can be worn (e.g., a smart watch) or implanted (e.g., a small patch in the neck or arm or another suitable site). FIG. 6 shows an example 600 of such a configuration where optical sources (such as LEDs) and acoustic/ultrasound sensors are interleaved together and integrated with the ASIC inside the helmet.

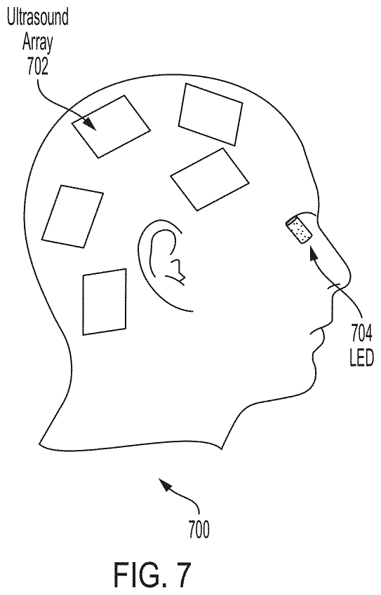

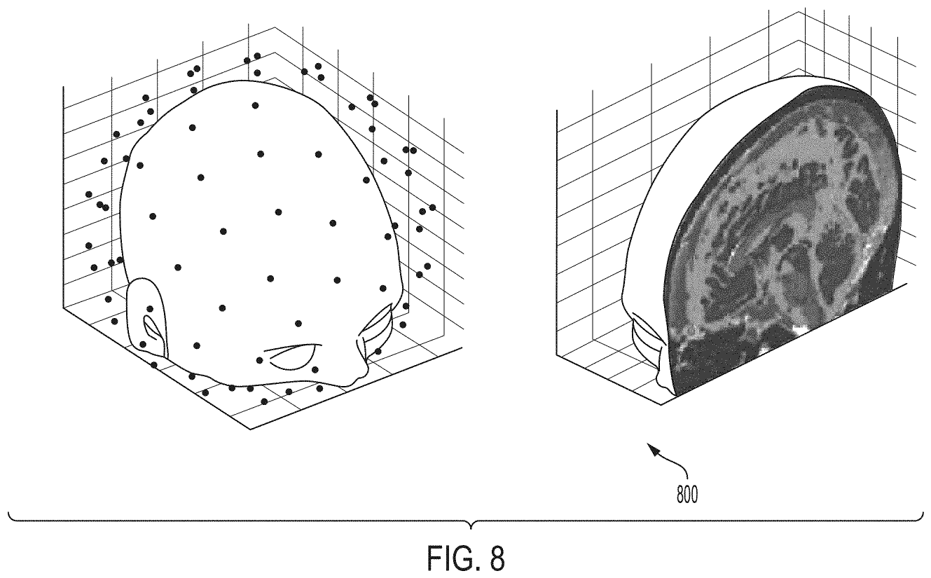

In some embodiments, a minimally invasive configuration includes an opto-acoustic device, where the acoustic sensors are integrated into a cap, attached to the scalp, or implanted under the scalp. The optical source may be implanted through the nasal canal, which provides a possible route for accessing the cranial cavity. FIG. 7 shows an example 700 of such a configuration where acoustic sensors 702 (e.g., an ultrasound array) are attached to the scalp of a person and optical source 704 (e.g., an LED) is implanted through the nasal canal of the person. The ultrasound detectors can be positioned around the head, e.g., as shown in example 800 in FIG. 8. This is advantageous in that the optical energy does not need to pass through the skull bone. This technique can be used to both detect and localize the source of the acoustic wave, depending on the configuration and population of the acoustic sensors that are placed over the head. In this technique: (1) the area (aperture) of the optical source, and thus the window of the illuminated area, may be limited; (2) the number of the wavelengths, and hence the ability of a broadband spectroscopy, may be limited.

In some embodiments, a minimally invasive configuration includes an opto-acoustic device, where the entire opto-acoustic device is implanted through the nasal canal. This technique may allow both optical and acoustic energy to get into/come out of the brain without facing the difficulty of passing through the skull. However, both acoustic and optical apertures, and thus the field of view, may be limited.

In some embodiments, when the sensor measures a substantial deviation of the oxygen saturation from its baseline value (e.g., as measured by averaging the measurements over a period of many hours), the sensor passes its information to a machine learning model, such as a statistical model, which uses the measured oxygen saturation level, and optionally other sensor readings, including one or more of EEG, EKG, EMG, heart-rate, skin conductance, or other similar physiological signals, to determine whether or not a seizure is likely to occur within the near future (e.g., less than half hour, less than one hour, or another suitable time period).

In some embodiments, the systems and methods described herein employ a statistical model for classification, which may include one or more sub-components such as convolutional neural networks, recurrent neural networks such as LSTMs and GRUs, linear SVMs, radial basis function SVMs, logistic regression, and various techniques from unsupervised learning such as variational autoencoders (VAE), generative adversarial networks (GANs) which are used to extract relevant features from the raw input data.

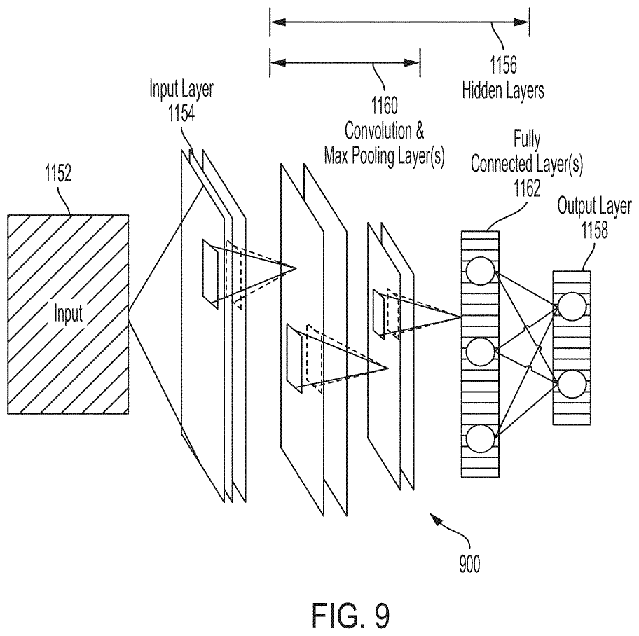

FIG. 9 shows a convolutional neural network 900 that may be used to implement a classification algorithm, in accordance with some embodiments of the technology described herein. The statistical model described herein may include the convolutional neural network 900, and additionally or alternatively another type of network, suitable for detecting and/or predicting whether the brain is exhibiting or will exhibit a symptom of a neurological disorder. For example, convolutional neural network 900 may be used to detect and/or predict a seizure in the brain. As shown, the convolutional neural network comprises an input layer 1154 configured to receive information about the input 1152 (e.g., a tensor), an output layer 1158 configured to provide the output (e.g., classifications in an n-dimensional representation space), and a plurality of hidden layers 1156 connected between the input layer 1154 and the output layer 1158. The plurality of hidden layers 1156 include convolution and pooling layers 1160 and fully connected layers 1162.

The input layer 1154 may be followed by one or more convolution and pooling layers 1160. A convolutional layer may comprise a set of filters that are spatially smaller (e.g., have a smaller width and/or height) than the input to the convolutional layer (e.g., the input 1152). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 1160 may be followed by fully connected layers 1162. The fully connected layers 1162 may comprise one or more layers each with one or more neurons that receives an input from a previous layer (e.g., a convolutional or pooling layer) and provides an output to a subsequent layer (e.g., the output layer 1158). The fully connected layers 1162 may be described as “dense” because each of the neurons in a given layer may receive an input from each neuron in a previous layer and provide an output to each neuron in a subsequent layer. The fully connected layers 1162 may be followed by an output layer 1158 that provides the output of the convolutional neural network. The output may be, for example, an indication of which class, from a set of classes, the input 1152 (or any portion of the input 1152) belongs to. The convolutional neural network may be trained using a stochastic gradient descent type algorithm or another suitable algorithm. The convolutional neural network may continue to be trained until the accuracy on a validation set (e.g., a held out portion from the training data) saturates or using any other suitable criterion or criteria.

It should be appreciated that the convolutional neural network shown in FIG. 9 is only one example implementation and that other implementations may be employed. For example, one or more layers may be added to or removed from the convolutional neural network shown in FIG. 9. Additional example layers that may be added to the convolutional neural network include: a pad layer, a concatenate layer, and an upscale layer. An upscale layer may be configured to upsample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output. As another example, in some embodiments, one or more convolutional, transpose convolutional, pooling, unpooling layers, and/or batch normalization may be included in the convolutional neural network. As yet another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect.

Any suitable optimization technique may be used for estimating neural network parameters from training data. For example, one or more of the following optimization techniques may be used: stochastic gradient descent (SGD), mini-batch gradient descent, momentum SGD, Nesterov accelerated gradient, Adagrad, Adadelta, RMSprop, Adaptive Moment Estimation (Adam), AdaMax, Nesterov-accelerated Adaptive Moment Estimation (Nadam), AMSGrad.

Convolutional neural networks may be employed to perform any of a variety of functions described herein. It should be appreciated that more than one convolutional neural network may be employed to make predictions in some embodiments.

### Using Opto-Acoustic Sensors for Monitoring Brain Conditions

In some aspects, the described opto-acoustic sensor may be used to monitor one or more brain conditions, described below, based on correlation between the oxygen content and these conditions.

In some aspects, the described opto-acoustic sensor may be used to measure Intracranial Pressure (ICP). Intracranial pressure (ICP) is the pressure inside the skull and thus in the brain tissue and cerebrospinal fluid (CSF). ICP is measured in millimeters of mercury (mmHg) and, at rest, is normally 7-15 mmHg for a supine adult. The body has various mechanisms by which it keeps the ICP stable, with CSF pressures varying by about 1 mmHg in normal adults through shifts in production and absorption of CSF. Changes in ICP may be attributed to volume changes in one or more of the constituents contained in the cranium. CSF pressure may be influenced by abrupt changes in intrathoracic pressure during coughing (intra-abdominal pressure), valsalva maneuver, and communication with the vasculature (venous and arterial systems).

In some aspects, the described opto-acoustic sensor may be used for detection and monitoring hemorrhage, including assessment of the clot volume and recurrence over time, assessment of the midline shift and brain compression, and guided insertion of catheters.

In some aspects, the described opto-acoustic sensor may be used for vasospasm detection and monitoring. Conventionally, bedside transcranial ultrasound is used for vasospasm detection. However, passing of ultrasound waves through the skull is still a limiting factor. Moreover, vasospasm can happen at any moment and patients are often critically ill and in a coma without a reliable physical exam.

### Using Opto-Acoustic Sensors for Neuro-Navigation

In some aspects, the described opto-acoustic sensor may be used to perform one or more instances of neuro-navigation, described below, based on correlation between the oxygen content and the targeted portion of the brain.

In some aspects, the described opto-acoustic sensor may be used to locate the ventricular system, required for/assisting in inserting ventricular drains, implanting CSF shunts.

In some aspects, the described opto-acoustic sensor may be used to perform precise puncture procedures for brain lesion biopsy, intracerebral abscess, and intracranial cyst puncture.

In some aspects, the described opto-acoustic sensor may be used for navigation of the ultrasound beam for focused ultrasound therapy.

Closed-Loop System for Brain Monitoring and/or Treatment

In some aspects, the inventors herein have developed a single, combined device to predict and suppress seizures, as part of a closed-loop, responsive system with machine learning. The closed-loop system may obtain feedback indicating brain state and, based on the feedback, apply one or more signals to preventatively or actively treat a condition indicated by the brain state. For example, a single CMUT device may be used as an ultrasound transmitter and receiver. Conventionally, such devices are used in pulse-echo mode, but the inventors have developed a novel application described herein where the ultrasound receiver may be used to perform photoacoustic sensing. The ultrasound transmitter may be used to send focused ultrasound radiation through the skull and into the brain to selectively activate and/or inhibit groups of neurons. While photoacoustic sensing has been described above, neural modulation using transcranial focused ultrasound is described below.

Neurons in the brain are sensitive to ultrasound. If ultrasound sequences are applied with properties including but not limited to certain carrier frequencies, pulse durations, pulse repetition frequencies, burst durations, and power levels, neurons may become more or less active (e.g., as measured by the rate at which they generate action potentials). The skull attenuates ultrasound in an amount which scales roughly exponentially with the carrier frequency. To obtain suitable transmission through the skull, it is therefore necessary to use carrier frequencies at, near, or below 500 kHz in some embodiments, and at, near or below 1 MHz in other embodiments. At such frequencies, significant levels of ultrasound radiation can pass through the skull and reach the brain tissue, which is significantly less attenuating. The brain tissue may defocus the ultrasound beam to some extent, but this has not proved to be an issue in experiments to date. By fixing an ultrasound transducer, for example a piezoelectric crystal or a CMUT array, to the scalp, ultrasound radiation is transmitted through the skull and to brain tissue. The radiating beam can be focused to a location within the brain at a resolution of several cubic millimeters (or a lower or higher resolution, if the application requires it) using either an acoustic lens on the transducer or by “phasing” the array (otherwise known as beamforming). This process may also be referred to as ultrasonic neuromodulation.

In both photoacoustic sensing and ultrasonic neuromodulation, the requirements for the acoustic waves are the same: the signals should be optimized for maximal transmission through the skull. In photoacoustic sensing, the described systems and methods record ultrasound signals that originate in the brain and reach the receiver located on the scalp by a path that crosses through several centimeters (or less) of brain tissue and the entire skull thickness. In using ultrasound for neural modulation, the described systems and methods transmit ultrasound signals originating at the scalp through the entire thickness of the skull and through a certain distance of brain tissue (e.g., on the order of 10 cm or less).

To meet the criteria outlined above, the inventors have designed a CMUT array that has significant transmission power and receiving sensitivity in the range of 500 kHz. Traditional CMUT arrays have difficulty operating at such low frequencies. To overcome these limitations, the inventors have designed CMUT arrays that operate in collapse mode, described further below. The CMUT design may be optimized for a combination of transmit and receive functionalities. Such a design is different than what would be obtained from optimizing for transmit or receive functionality alone.

In collapse mode of operation, the CMUT cells are designed such that part of the top plate is in physical contact with the substrate, yet electrically isolated with a dielectric, during normal operation. The transmit and receive sensitivities of the CMUT are further enhanced thus providing a superior solution for ultrasound transducers. In short, the CMUT is a high electric field device, and if one can control the high electric field from issues like charging and breakdown, then one has an ultrasound transducer with superior bandwidth and sensitivity, amenable for integration with electronics, manufactured using traditional integrated circuits fabrication technologies with all its advantages, and can be made flexible for wrapping around a cylinder or even over human tissue. While the CMUT operation is described with respect to the collapse mode, a standard mode of operation or another suitable mode of operation for the CMUT may be equally applicable to some or all embodiments described herein.

As an example, the device is operated as follows: At a frequency of about once per second, the optical transmitter transmits a pulsed-sequence of infrared radiation, with pulse repetition frequency at or around 500 kHz. The CMUT receiver then records the ensuing ultrasound response, and determines the oxygen saturation in the brain tissue located in a predetermined volume corresponding to a particular artery, vein, capillary, or tissue volume of interest. In an example, in the manner described above with respect to FIG. 9, this oxygen saturation measurement is passed as input into a machine learning model, such as a statistical model, which optionally also receives input from other sensor readings included but not limited to EEG, EKG, EMG, heart-rate and skin conductance. The machine learning model then determines whether or not a seizure is likely to occur in the near future. If the machine learning model determines that a seizure is likely to occur in the near future, a signal may be sent to the sensor to switch the mode of the sensor from photoacoustic sensing to ultrasound stimulation. At this point, the array begins sending ultrasound radiation at a carrier frequency at or around 500 kHz into the brain to inhibit the seizure or pre-seizure activity.

In some embodiments, the above described device may be configured in a manner similar to the opto-acoustic sensor illustrated in FIG. 1. The opto-acoustic sensor may feature an optical transmitter (operating in the infrared or visible range), such as an LED array, a laser, or a laser diode, or similar devices, and an acoustic receiver, such as a CMUT or a piezoelectric crystal or similar devices. In some embodiments, the device may include separate ultrasound detection and suppression arrays. In some embodiments, the device may include one array configured for both ultrasound detection and suppression.

In some embodiments, the closed-loop system obtains feedback from the brain using a metric other than, or in addition to, oxygen saturation in order to determine when to transmit ultrasound radiation to the brain, e.g., to suppress a likely occurrence of a seizure in the brain. For example, the closed-loop system may obtain EEG data and/or functional MRI data and use solely EEG data and/or the functional MRI data, or combine the oxygen saturation and the EEG data and/or functional MRI data (and/or other data), in order to determine when to transmit ultrasound radiation to the brain.

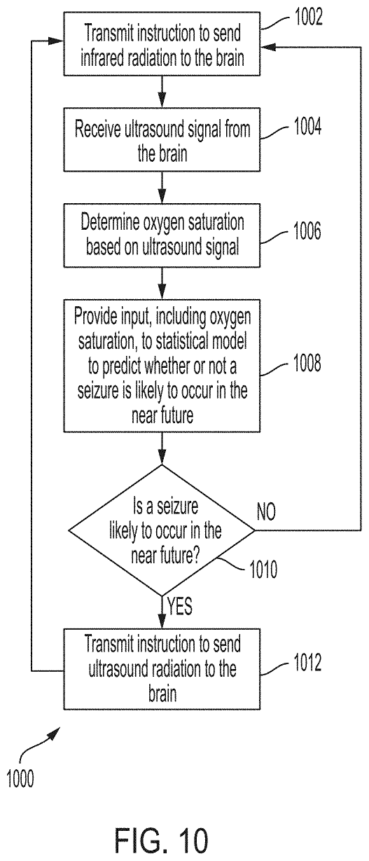

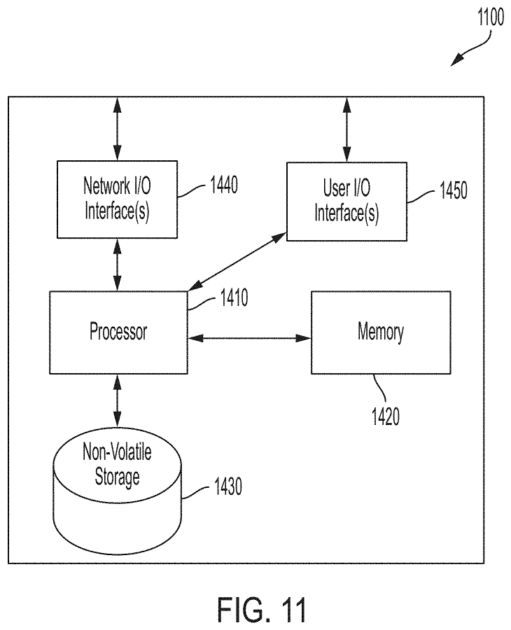

FIG. 10 shows an illustrative flow diagram for a process for brain monitoring and treatment, in accordance with some embodiments of the technology described herein. Process 1000 illustrated in FIG. 10 may be implemented on a processor, e.g., processor 1410 illustrated with respect to FIG. 11. The processor may be in communication with a sensor, e.g., as described with respect to FIGS. 1-8.

At step 1002, the processor transmits an instruction to send infrared radiation to the brain. For example, the processor may generate and transmit the instruction to an optical transmitter to send infrared radiation to the brain of a person. The infrared radiation may be sent before or after ultrasound radiation is sent to the brain, e.g., as described with respect to step 1012.

At step 1004, the processor receives an ultrasound signal from the brain. The ultrasound signal may be indicative of a level of oxygen saturation. As described above, a magnitude of the acoustic response, such as the ultrasound signal, may be used to deduce the concentration of the absorbing compound in the sample, such as the oxygen saturation.

At step 1006, the processor determines oxygen saturation of the brain tissue based on the ultrasound signal. The oxygen saturation of the brain tissue may be indicative of neural activity in the brain, such as a seizure. In some embodiments, the processor may determine another metric in addition to, or instead of, oxygen saturation in order to predict a likelihood of seizure, e.g., EEG or another suitable metric.

At step 1008, the processor provides input, e.g., including the oxygen saturation or another metric, to a statistical model to predict whether or not a seizure is likely to occur for the person in the near future (e.g., in the next half hour, one hour, or another suitable time period). For example, the processor may provide the input to statistical model 900 (FIG. 9) to predict the likelihood of seizure in the near future.

At step 1010, the processor receives the output from the statistical model and proceeds to step 1012 based on the output indicating a seizure is likely to occur in the near future. Otherwise, e.g., based on the output indicating a seizure is not likely to occur in the near future, the processor returns to step 1002 to continue monitoring the brain of the person.

At step 1012, the processor transmits an instruction to send ultrasound radiation to the brain of the person. For example, the ultrasound radiation may help prevent and/or treat a seizure likely to occur or occurring to the person. The ultrasound radiation may be sent before or after infrared radiation is sent to the brain, e.g., as described with respect to step 1002.

### Example Computer Architecture

An illustrative implementation of a computer system 1100 that may be used in connection with any of the embodiments of the technology described herein is shown in FIG. 11. The computer system 1100 includes one or more processors 1410 and one or more articles of manufacture that comprise non-transitory computer-readable storage media (e.g., memory 1420 and one or more non-volatile storage media 1430). The processor 1410 may control writing data to and reading data from the memory 1420 and the non-volatile storage device 1430 in any suitable manner, as the aspects of the technology described herein are not limited in this respect. To perform any of the functionality described herein, the processor 1410 may execute one or more processor-executable instructions stored in one or more non-transitory computer-readable storage media (e.g., the memory 1420), which may serve as non-transitory computer-readable storage media storing processor-executable instructions for execution by the processor 1410.

Computing device 1100 may also include a network input/output (I/O) interface 1440 via which the computing device may communicate with other computing devices (e.g., over a network), and may also include one or more user I/O interfaces 1450, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing,” “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

While some aspects and/or embodiments described herein are described with respect to treating seizures for epilepsy, these aspects and/or embodiments may be equally applicable to monitoring and/or treating symptoms for any suitable neurological disorder. Any limitations of the embodiments described herein are limitations only of those embodiments, and are not limitations of any other embodiments described herein.

What is claimed is:

1. A closed-loop system for monitoring and/or treating brain function of a person, comprising:
a capacitive micromachined ultrasound transducer (CMUT) device that transmits ultrasound radiation to the brain of the person and receives and detects an ultrasound signal generated in the brain of the person; and
a processor that assesses the received ultrasound signal to determine brain function and controls further transmission of ultrasound radiation from the CMUT device based on the determined brain function.

2. The system as claimed in claim 1, wherein assessing the received ultrasound signal to determine brain function comprises assessing a likelihood of a seizure of the person, and controlling further transmission of ultrasound radiation from the CMUT device comprises controlling further transmission of ultrasound radiation from the CMUT device when the likelihood is greater than a certain threshold.

3. The system as claimed in claim 1, wherein the system comprises an array of CMUT devices.

4. The system as claimed in claim 1, wherein the CMUT device transmits at, near or below 1 MHz.

5. The system as claimed in claim 1, wherein the processor implements a statistical model to predict, based on the received ultrasound signal, whether a seizure will occur in the brain.

6. The system as claimed in claim 1, wherein the system comprises an optical transmitter that transmits to the brain infrared radiation.

7. The system as claimed in claim 6, wherein the CMUT device receives and detects the ultrasound signal generated in the brain in response to the infrared radiation.

8. The system as claimed in claim 1, wherein determining brain function comprises determining oxygen saturation of the brain based on the received ultrasound signal, wherein the oxygen saturation of the brain is indicative of neural activity in the brain.

9. A method of monitoring and/or treating brain function of a person, comprising:
transmitting with a CMUT device ultrasound radiation to the brain of the person;
receiving an ultrasound signal generated in the brain of the person;
determining brain function based on the received ultrasound signal; and
controlling further transmission of ultrasound radiation from the CMUT device based on the determined brain function.

10. The method as claimed in claim 9, wherein assessing the received ultrasound signal to determine brain function comprises assessing a likelihood of a seizure of the person, and controlling further transmission of ultrasound radiation from the CMUT device comprises controlling further transmission of ultrasound radiation from the CMUT device when the likelihood is greater than a certain threshold.

11. The method as claimed in claim 9, wherein the determining is performed by a processor implementing a statistical model to predict, based on the received ultrasound signal, whether a seizure will occur in the brain.

12. The method as claimed in claim 9, wherein the method further comprises transmitting to the brain infrared radiation.

13. The method as claimed in claim 12, wherein the received ultrasound signal is generated in the brain of the person in response to the infrared radiation.

14. The method as claimed in claim 9, wherein determining brain function comprises determining oxygen saturation of the brain based on the received ultrasound signal.

15. The method as claimed in claim 14, wherein the oxygen saturation of the brain is indicative of neural activity in the brain.
